(19) United States
(12) Patent Application Publication
(10) Pub. No.: US 2026/0185110 A1
(43) Pub. Date: Jul. 2, 2026

(54) GENETICALLY MODIFIED MICROORGANISM THAT ENHANCES PRODUCTION OF POLYKETIDES OR DERIVATIVES THEREOF AND METHOD FOR PRODUCING POLYKETIDES OR DERIVATIVES THEREOF

(71) Applicant: INDUSTRIAL TECHNOLOGY RESEARCH INSTITUTE, Hsinchu (TW)

(72) Inventors: Ke-Ming LIANG, Hsinchu City (TW); Chia-Chun CHEN, Hsinchu City (TW); Ya-Lin LIN, Hsinchu City (TW); Jie-Len HUANG, Hsinchu City (TW); Pei-Ching CHANG, Zhubei City (TW)

(73) Assignee: INDUSTRIAL TECHNOLOGY RESEARCH INSTITUTE, Hsinchu (TW)

(21) Appl. No.: 19/001,967

(22) Filed: Dec. 26, 2024

(51) Int. Cl. C12N 15/70; C12N 1/20; C12N 9/10; C12N 15/52; C12P 7/42; C12P 17/06; C12R 1/19

(52) U.S. Cl. CPC C12N 15/70; C12N 1/20; C12N 9/1029; C12N 9/1096; C12N 9/13; C12N 15/52; C12P 7/42; C12P 17/06; C12R 2001/19; C12Y 203/01008; C12Y 206/01018; C12Y 208/04001

(57) ABSTRACT

A genetically modified microorganism for increasing the production of a polyketide compound or a derivative thereof is provided, including any one or both of the following genetic modifications: (a) a deleted endogenous pta gene encoding a phosphate acetyltransferase, wherein an expression level of the phosphate acetyltransferase of the deleted endogenous pta gene is lower than an expression level of its wild type; and (b) an added first exogenous nucleotide sequence and second exogenous nucleotide sequence, wherein the first exogenous nucleotide sequence encodes a transaminase and the second exogenous nucleotide sequence encodes a reductase. A method for producing a polyketide compound or a derivative thereof using the aforementioned genetically modified microorganism is also provided.

### INCORPORATION BY REFERENCE OF SEQUENCE LISTING

A sequence listing submitted as an ST.26 XML format is incorporated herein by reference. The file containing the sequence listing is named “9044C-P240197700-US_Seq_F”; its date of creation was Dec. 25, 2024; and its size is 35 kilobytes.

### TECHNICAL FIELD

The present disclosure relates to a genetically modified microorganism and a method for producing polyketide compounds or derivatives thereof using the same.

### BACKGROUND

Chemical synthesis is usually highly dependent on petrochemical raw materials and has high carbon emissions. Following the global trend of reducing carbon emissions and seeking to achieve net zero, biosynthesis technology has gradually gained attention because it is in line with environmental protection dictates.

Acetyl-CoA and malonyl-CoA are important initial reactants in the production pathway of polyketides. In the metabolic pathway for the production of acetyl-CoA, phosphate acetyltransferase expressed by the pta gene consumes acetyl-CoA to produce acetate, thereby reducing the concentration of acetyl-CoA in organisms. On the other hand, in the metabolic pathway for the production of malonyl-CoA, the enzyme activity of the carboxylase involved in the catalytic reaction will be feedback-inhibited by the product malonyl-CoA, and at the same time, the energy molecule adenosine triphosphate (ATP) will also be consumed, resulting in the inability to increase the concentration of malonyl-CoA in organisms. Low concentrations of acetyl-CoA and malonyl-CoA will indirectly affect the carbon flow of the polyketide biosynthesis pathway, thereby limiting the production of polyketide compounds and derivatives thereof.

For example, carmine, a pigment widely used in high-priced cosmetics, is one of the common derivatives of polyketide compounds. Since carmine contains a complex structure of an anthraquinone polyketide compound connected to a glucose molecule, it is quite difficult to prepare it by chemical synthesis and the yield is not high. Furthermore, the mainstream method of extracting carmine from cochineal is difficult to mass produce due to factors such as the long growth cycle of cochineal, geographical and climatic restrictions, and complicated extraction steps.

As mentioned above, developing a biosynthetic method that can improve the production efficiency of polyketide compounds or their derivatives, thereby enhancing the market competitiveness of related products, is still one of the research goals currently being devoted to in the industry.

### SUMMARY

In accordance with some embodiments of the present disclosure, a genetically modified microorganism for increasing the production of a polyketide compound or a derivative thereof is provided, including any one or both of the following genetic modifications: (a) a deleted endogenous pta gene encoding a phosphate acetyltransferase, wherein an expression level of the phosphate acetyltransferase of the deleted endogenous pta gene is lower than an expression level of its wild type; and (b) an added first exogenous nucleotide sequence and second exogenous nucleotide sequence, wherein the first exogenous nucleotide sequence encodes a transaminase and the second exogenous nucleotide sequence encodes a reductase.

In accordance with some other embodiments of the present disclosure, a method for producing a polyketide compound or a derivative thereof is provided, including the following steps: (a) providing an aforementioned genetically modified microorganism; (b) providing a first culture medium, inoculating the genetically modified microorganism into the first culture medium, and culturing the genetically modified microorganism at a temperature of 28° C. to 37° C. for 12 hours to 24 hours; (c) inoculating the first culture medium containing the cultured genetically modified microorganism into a second culture medium, and culturing the genetically modified microorganism at a temperature of 28° C. to 37° C. for 24 hours to 80 hours to obtain the second culture medium containing a polyketide compound or a derivative thereof; and (d) separating the polyketide compound or the derivative thereof from the second culture medium of step (c).

In accordance with still some other embodiments of the present disclosure, a novel genetically modified Escherichia coli strain is provided, and its deposit number is BCRC 940701. An endogenous pta gene of the novel genetically modified Escherichia coli strain is deleted and it includes a first exogenous nucleotide sequence and a second exogenous nucleotide sequence. The endogenous pta gene encodes a phosphate acetyltransferase, the first exogenous nucleotide sequence encodes a transaminase, and the second exogenous nucleotide sequence encodes a reductase.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 shows a biosynthetic pathway of carminic acid constructed by a genetically modified microorganism in accordance with some embodiments of the present disclosure;

FIG. 2 shows a plasmid construction map of a genetically modified microorganism in accordance with some embodiments of the present disclosure;

FIG. 3 shows a high performance liquid chromatography (HPLC) analysis of products produced by a genetically modified microorganism via a polyketide synthesis metabolic pathway in accordance with some embodiments of the present disclosure;

FIG. 4A and FIG. 4B respectively show the results of testing the effect of pta gene deletion on the production of acetic acid and polyketide derivatives (flavokermesic acid) of the strains in accordance with some embodiments of the present disclosure, wherein the “Control group” refers to a strain in which the pta gene is not deleted, and the “pta gene deleted” refers to a genetically modified microorganism strain in which the pta gene is deleted, and both the “Control group” and the “pta gene deleted” described herein carry the plasmid of exogenous genes required for expressing the polyketide metabolic pathway in the strains;

FIG. 5 shows the effect of substitution of carboxylase with transaminase and/or reductase on the yield of polyketide derivatives (flavokermesic acid) of the strains in accordance with some embodiments of the present disclosure, wherein the “Control group” refers to a strain in which the gene of carboxylase is not substituted with the gene of transaminase and/or reductase, the “Substitution with transaminase alone” refers to a genetically modified microorganism strain in which the gene of carboxylase is substituted with the gene of transaminase, the “Substitution with reductase alone” refers to a genetically modified microorganism strain in which the gene of carboxylase is substituted with the gene of reductase, and the “Substitution with both transaminase and reductase” refers to a genetically modified microorganism strain in which the gene of carboxylase is substituted with the genes of transaminase and reductase, and the “Control group”, “Substitution with transaminase alone”, “Substitution with reductase alone” and “Substitution with both transaminase and reductase” described herein all carry the plasmid of exogenous genes required for expressing the polyketide metabolic pathway in the strains;

FIG. 6 shows the effects of substitution of carboxylase with transaminase and reductase and pta gene deletion on the yield of polyketide derivatives (flavokermesic acid) of the strains in accordance with some embodiments of the present disclosure, wherein the “Control group” refers to a strain in which the pta gene is not deleted and the gene of carboxylase is not substituted with the gene of transaminase and/or reductase, and the “Substitution with both transaminase and reductase+pta gene deletion” refers to a genetically modified microorganism strain in which the gene of carboxylase is substituted with the genes of both transaminase and reductase and the pta gene is deleted, and both the “Control group” and the “Substitution with both transaminase and reductase+pta gene deletion” described herein carry the plasmid of exogenous genes required for expressing the polyketide metabolic pathway in the strains.

### DETAILED DESCRIPTION

The genetically modified microorganism for increasing the production of a polyketide compound or a derivative thereof and a method for producing a polyketide compound or a derivative thereof of the present disclosure are described in detail in the following description. It should be understood that in the following detailed description, for purposes of explanation, numerous specific details and embodiments are set forth in order to provide a thorough understanding of the present disclosure. The specific elements and configurations described in the following detailed description are set forth in order to clearly describe the present disclosure. It will be apparent that the exemplary embodiments set forth herein are used merely for the purpose of illustration and not the limitations of the present disclosure.

Unless defined otherwise, all technical and scientific terms used herein have the same meaning as commonly understood by one of ordinary skill in the art to which this disclosure belongs. It should be appreciated that, in each case, the term, which is defined in a commonly used dictionary, should be interpreted as having a meaning that conforms to the relative skills of the present disclosure and the background or the context of the present disclosure, and should not be interpreted in an idealized or overly formal manner unless so defined. In order to make the content of the present disclosure easier to understand, the following definitions of terms and words are provided.

In the biological world, acetyl coenzyme A (acetyl-CoA) and malonyl coenzyme A (malonyl-CoA) are important metabolic raw materials for the synthesis of polyketide compounds. In accordance with some embodiments of the present disclosure, the endogenous pta gene is deleted in the genetically modified microorganism provided, thereby blocking the metabolic pathway that consumes acetyl-CoA to produce acetate, reducing the conversion and use of acetyl-CoA, and instead entering the polyketide metabolic pathway, thereby increasing the production of polyketide compounds or their related derivatives. In accordance with some embodiments of the present disclosure, the provided genetically modified microorganism utilizes a combination of transaminase and reductase to replace carboxylase. The production of malonyl-CoA in this manner is not affected by feedback inhibition of enzyme activity. Therefore, the concentration of malonyl-CoA in the microorganism can be increased, thereby increasing the production of polyketide compounds or their related derivatives.

In accordance with the embodiments of the present disclosure, a genetically modified microorganism for increasing the production of polyketide compounds or their derivatives is provided, including any one or both of the following genetic modifications: (a) a deleted endogenous pta gene, the pta gene encodes phosphate acetyltransferase, and the expression level of the phosphate acetyltransferase of the deleted endogenous pta gene is lower than that of its wild type; and (b) an added first exogenous nucleotide sequence and second exogenous nucleotide sequence, the first exogenous nucleotide sequence encodes a transaminase, and the second exogenous nucleotide sequence encodes a reductase. In accordance with some embodiments, the genetically modified microorganism comprises a genetic modification that deletes an endogenous pta gene. In accordance with some embodiments, the genetically modified microorganism includes a genetic modification of adding a first exogenous nucleotide sequence and a second exogenous nucleotide sequence. In accordance with some embodiments, the genetically modified microorganism includes a genetic modification of deleting an endogenous pta gene and a genetic modification of adding a first exogenous nucleotide sequence and a second exogenous nucleotide sequence.

In accordance with some embodiments, the source of genetically modified microorganisms includes bacteria. In accordance with some embodiments, the aforementioned bacteria may include Escherichia coli, Corynebacterium glutamicum, Bacillus subtilis, Pseudomonas putida, Yarrowia lipolytica, Saccharomyces cerevisiae or Pichia pastoris, but it is not limited thereto. In accordance with some embodiments, the aforementioned Escherichia coli may include Escherichia coli BL21, K12, BW25113, DH5a, XL1-blue, W3110 or other suitable strains, but it is not limited thereto.

In accordance with some embodiments, the phosphate acetyltransferase encoded by the pta gene may include the amino acid sequence shown in SEQ ID NO: 1. In accordance with some embodiments, the pta gene may include a nucleotide sequence having at least 85% sequence similarity to SEQ ID NO: 2, for example, it may include a nucleotide sequence having at least 88%, 90%, 92%, 95%, 98% or 99% sequence similarity to SEQ ID NO: 2, but it is not limited thereto. In accordance with some embodiments, the pta gene may include a nucleotide sequence as shown in SEQ ID NO: 2. In accordance with some embodiments, the pta gene may be a nucleotide sequence as shown in SEQ ID NO: 2.

In particular, in the genetically modified microorganism in which the endogenous pta gene is deleted, the expression level of phosphate acetyltransferase is lower than that of the wild type of the genetically modified microorganism. As mentioned above, the pta gene encodes the phosphate acetyltransferase, which consumes acetyl-CoA in the body to generate acetate, thereby reducing the concentration of acetyl-CoA. Therefore, the deletion of the endogenous pta gene can block the metabolic pathway by which the genetically modified microorganism consumes acetyl-CoA to produce acetate, thereby increasing the concentration of acetyl-CoA in the genetically modified microorganism.

Furthermore, the first exogenous nucleotide sequence encodes a transaminase. In accordance with some embodiments, the transaminase may include β-alanine-pyruvate transaminase (BauA). In accordance with some embodiments, the transaminase may include an amino acid sequence as shown in SEQ ID NO: 3. In accordance with some embodiments, the first exogenous nucleotide sequence encoding the transaminase may include a nucleotide sequence having at least 85% sequence similarity to SEQ ID NO: 4, for example, it may include a nucleotide sequence having at least 88%, 90%, 92%, 95%, 98% or 99% sequence similarity to SEQ ID NO: 4, but it is not limited thereto. In accordance with some embodiments, the first exogenous nucleotide sequence encoding the transaminase may include a nucleotide sequence as shown in SEQ ID NO: 4. In accordance with some embodiments, the first exogenous nucleotide sequence encoding the transaminase may be a nucleotide sequence as shown in SEQ ID NO: 4.

In accordance with some embodiments, the first exogenous nucleotide sequence may be linked to a first promoter. In accordance with some embodiments, the first promoter may include T7 promoter, lac promoter, Ptac promoter, araBAD promoter or other suitable promoters, but it is not limited thereto.

Moreover, the second exogenous nucleotide sequence encodes a reductase. In accordance with some embodiments, the reductase may include methyl coenzyme M reductase operon protein C (MCR-C). In accordance with some embodiments, the reductase may include an amino acid sequence as shown in SEQ ID NO: 5. In accordance with some embodiments, the second exogenous nucleotide sequence encoding the reductase may include a nucleotide sequence having at least 85% sequence similarity to SEQ ID NO: 6, for example, it may include a nucleotide sequence having at least 88%, 90%, 92%, 95%, 98% or 99% sequence similarity to SEQ ID NO: 6, but it is not limited thereto. In accordance with some embodiments, the second exogenous nucleotide sequence encoding the reductase may include a nucleotide sequence as shown in SEQ ID NO: 6. In accordance with some embodiments, the second exogenous nucleotide sequence encoding the reductase may be a nucleotide sequence as shown in SEQ ID NO: 6.

In accordance with some embodiments, the second exogenous nucleotide sequence and the first exogenous nucleotide sequence may be connected to the same first promoter. In accordance with some other embodiments, the second exogenous nucleotide sequence may be linked to a second promoter different from the first promoter. In accordance with some embodiments, the second promoter may include T7 promoter, lac promoter, Ptac promoter, araBAD promoter or other suitable promoters, but it is not limited thereto.

In vivo, carboxylase can catalyze the chemical reaction of converting acetyl-CoA into malonyl-CoA. However, the activity of carboxylase is inhibited by the product malonyl-CoA, which results in the inability to generate high concentrations of malonyl-CoA in vivo. In particular, in the genetically modified microorganism provided in the embodiments of the present disclosure, the combination of transaminase and reductase can replace the carboxylase in the organism, thereby converting acetyl-CoA into malonyl-CoA without being affected by feedback inhibition of enzyme activity, thereby increasing the concentration of malonyl-CoA in the genetically modified microorganism.

In addition, in accordance with some embodiments, the genetically modified microorganism may further include (c) an added third exogenous nucleotide sequence, and the third exogenous nucleotide sequence encodes an enzyme related to carmine synthesis. In accordance with some embodiments, the enzymes related to carmine synthesis may include at least one of the following: cyclase ZhuI, aromatase ZhuJ, type II polyketide synthase complex AntDEFGB, hydroxylase DnrF^{P217K}, glucosyltransferase GtCGT^{V93Q/Y193F}, glucosyltransferase UGT2, monooxygenase AptC and 4′-phosphopantetheinyl transferase NpgA, but it is not limited thereto.

In accordance with some embodiments, the third exogenous nucleotide sequence encoding the cyclase ZhuI may include a nucleotide sequence having at least 85% sequence similarity to SEQ ID NO: 7, for example, it may include a nucleotide sequence having at least 88%, 90%, 92%, 95%, 98% or 99% sequence similarity to SEQ ID NO: 7, but it is not limited thereto. In accordance with some embodiments, the third exogenous nucleotide sequence encoding the cyclase ZhuI may include a nucleotide sequence as shown in SEQ ID NO: 7. In accordance with some embodiments, the third exogenous nucleotide sequence encoding the cyclase ZhuI may be a nucleotide sequence as shown in SEQ ID NO: 7.

In accordance with some embodiments, the third exogenous nucleotide sequence encoding the aromatase ZhuJ may include a nucleotide sequence having at least 85% sequence similarity to SEQ ID NO: 8, for example, it may include a nucleotide sequence having at least 88%, 90%, 92%, 95%, 98% or 99% sequence similarity to SEQ ID NO: 8, but it is not limited thereto. In accordance with some embodiments, the third exogenous nucleotide sequence encoding the aromatase ZhuJ may include a nucleotide sequence as shown in SEQ ID NO: 8. In accordance with some embodiments, the third exogenous nucleotide sequence encoding the aromatase ZhuJ may be a nucleotide sequence as shown in SEQ ID NO: 8.

In accordance with some embodiments, the third exogenous nucleotide sequence encoding the polyketide synthase AntDEFGB may include a nucleotide sequence having at least 85% sequence similarity to at least one of SEQ ID NO: 9, SEQ ID NO: 10, SEQ ID NO: 11, SEQ ID NO: 12, and SEQ ID NO: 13, for example, it may include a nucleotide sequence having at least 88%, 90%, 92%, 95%, 98%, or 99% sequence similarity to at least one of SEQ ID NO: 9, SEQ ID NO: 10, SEQ ID NO: 11, SEQ ID NO: 12, and SEQ ID NO: 13, but it is not limited thereto. In accordance with some embodiments, the third exogenous nucleotide sequence encoding the polyketide synthase AntDEFGB may include a nucleotide sequence as shown in SEQ ID NO: 9, SEQ ID NO: 10, SEQ ID NO: 11, SEQ ID NO: 12, and SEQ ID NO: 13. In accordance with some embodiments, the third exogenous nucleotide sequence encoding the polyketide synthase AntDEFGB may be a nucleotide sequence as shown in SEQ ID NO: 9, SEQ ID NO: 10, SEQ ID NO: 11, SEQ ID NO: 12, and SEQ ID NO: 13. In detail, the nucleotide sequences shown in SEQ ID NO: 9, SEQ ID NO: 10, SEQ ID NO: 11, SEQ ID NO: 12 and SEQ ID NO: 13 can respectively encode AntD, AntE, AntF, AntG and AntB units of polyketide synthase, and can be co-expressed and polymerized as the polyketide synthase.

In accordance with some embodiments, the third exogenous nucleotide sequence encoding the hydroxylase DnrF^{P217K} may include a nucleotide sequence having at least 85% sequence similarity to SEQ ID NO: 14, for example, it may include a nucleotide sequence having at least 88%, 90%, 92%, 95%, 98% or 99% sequence similarity to SEQ ID NO: 14, but it is not limited thereto. In accordance with some embodiments, the third exogenous nucleotide sequence encoding the hydroxylase DnrF^{P217K} may include a nucleotide sequence as shown in SEQ ID NO: 14. In accordance with some embodiments, the third exogenous nucleotide sequence encoding the hydroxylase DnrF^{P217K} may be a nucleotide sequence as shown in SEQ ID NO: 14.

In accordance with some embodiments, the third exogenous nucleotide sequence encoding the glucosyltransferase GtCGT^{V93Q/Y193F} may include a nucleotide sequence having at least 85% sequence similarity to SEQ ID NO: 15, for example, it may include a nucleotide sequence having at least 88%, 90%, 92%, 95%, 98% or 99% sequence similarity to SEQ ID NO: 15, but it is not limited thereto. In accordance with some embodiments, the third exogenous nucleotide sequence encoding the glucosyltransferase GtCGT^{V93Q/Y193F} may include a nucleotide sequence as shown in SEQ ID NO: 15. In accordance with some embodiments, the third exogenous nucleotide sequence may be the nucleotide sequence shown in SEQ ID NO: 15.

In accordance with some embodiments, the third exogenous nucleotide sequence may be connected to a third promoter. In accordance with some embodiments, the third promoter may include T7 promoter, lac promoter, Ptac promoter, araBAD promoter or other suitable promoters, but it is not limited thereto.

As described above, the genetically modified microorganism provided in the embodiments of the present disclosure can increase the production of polyketide compounds or their derivatives. In accordance with some embodiments, the polyketide compound may include flavokermesic acid, but it is not limited thereto. In accordance with some embodiments, compared to its wild type, the production of flavokermesic acid in the genetically modified microorganism provided by the embodiments of the present disclosure can be increased by 1.5 to 5 times, for example, 2 times, 2.5 times, 3 times, 3.5 times, 4 times or 4.5 times, but it is not limited thereto. In accordance with some embodiments, the derivative of the polyketide compound may include carminic acid, but it is not limited thereto. In accordance with some embodiments, compared to its wild type, the production of carminic acid in the genetically modified microorganism provided by the embodiments of the present disclosure can be increased by 1.5 to 5 times, for example, 2 times, 2.5 times, 3 times, 3.5 times, 4 times or 4.5 times, but it is not limited thereto.

In accordance with some embodiments, the aforementioned genetically modified microorganism for increasing the production of polyketide compounds or their derivatives has a deposit number of BCRC 940701.

Furthermore, in accordance with some embodiments of the present disclosure, a novel genetically modified Escherichia coli strain is provided, and its deposit number is BCRC 940701. The endogenous pta gene of the aforementioned novel genetically modified Escherichia coli strain is deleted and includes a first exogenous nucleic acid sequence and a second exogenous nucleic acid sequence. The endogenous pta gene encodes a phosphate acetyltransferase, the first exogenous nucleic acid sequence encodes a transaminase, and the second exogenous nucleic acid sequence encodes a reductase.

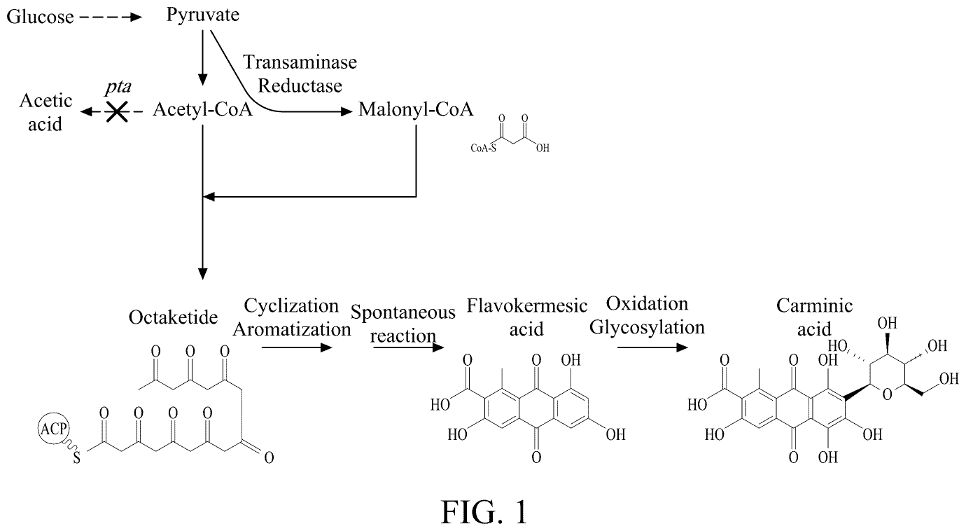

Specifically, please refer to FIG. 1, which shows a biosynthetic pathway of carminic acid constructed by a genetically modified microorganism in accordance with some embodiments of the present disclosure. In genetically modified microorganisms, glucose is decomposed into pyruvate via glycolysis, and pyruvate can produce acetyl-CoA via oxidative decarboxylation. Since the endogenous pta gene of the genetically modified microorganism is deleted, the metabolic pathway that consumes acetyl-CoA to produce acetate can be blocked, thereby increasing the concentration of acetyl-CoA in the genetically modified microorganism. On the other hand, pyruvate can be converted into malonyl-CoA through the action of transaminase and reductase. The combination of transaminase and reductase can replace the action of carboxylase. The production of malonyl-CoA in this manner is not affected by feedback inhibition of enzyme activity. In this way, malonyl-CoA is converted and produced without being affected by the feedback inhibition of the enzymatic activity of carboxylase by its product malonyl-CoA. Therefore, the concentration of malonyl-CoA in the genetically modified microorganism can be increased. Reducing the consumption of acetyl-CoA and increasing the concentration of malonyl-CoA can increase the carbon flow in the polyketide synthesis metabolic pathway, thereby increasing the production of polyketide compounds and their related derivatives accordingly. Specifically, acetyl-CoA and malonyl-CoA can then be condensed by polyketide synthase (e.g., AntDEFGB) to synthesize octaketide, which is then converted into flavokermesic acid by the action of cyclase (e.g., ZhuI) and aromatase (e.g., ZhuJ), and then converted into kermesic acid by the action of hydroxylase (e.g., DnrF^{P217K}), and finally converted into carminic acid by the action of glucosyltransferase (e.g., GtCGT^{V93Q/Y193F}). Carmine can be obtained by adding aluminum salt or calcium salt to carminic acid.

In addition, in accordance with the embodiments of the present disclosure, a method for producing a polyketide compound or a derivative thereof is also provided, which includes the steps of (a) providing the aforementioned genetically modified microorganism for increasing the production of polyketide compounds or derivatives thereof; and (b) providing a first culture medium, inoculating the genetically modified microorganism into the first culture medium, and culturing the genetically modified microorganism at a temperature of 28° C. to 37° C. for 12 hours to 24 hours.

In accordance with some embodiments, the first culture medium may include LB medium (Lysogeny broth, LB), NB medium, M9 medium, TB medium or a combination thereof, but it is not limited thereto. In accordance with some embodiments, the first culture medium may include appropriate antibiotics, so that plasmids can be selected based on the antibiotics they tolerate. In accordance with some embodiments, the pH value of the first culture medium may be between pH 6 and pH 8, for example, pH 6.2, pH 6.5, pH 6.8, pH 7, pH 7.2, pH 7.5 or pH 7.8, but it is not limited thereto. In accordance with some embodiments, the temperature for culturing the genetically modified microorganism in the first culture medium may be, for example, 28° C., 29° C., 30° C., 31° C., 32° C., 33° C., 34° C., 35° C., 36° C. or 37° C., but it is not limited thereto. Furthermore, in accordance with some embodiments, the time period for culturing the genetically modified microorganism in the first culture medium may be 12 hours, 13 hours, 14 hours, 15 hours, 16 hours, 17 hours, 18 hours, 19 hours, 20 hours, 21 hours, 22 hours, 23 hours or 24 hours, but it is not limited thereto.

Furthermore, the method for producing a polyketide compound or a derivative thereof may include the step (c) inoculating the first culture medium containing the cultured genetically modified microorganism into a second culture medium, and culturing the genetically modified microorganism at a temperature of 28° C. to 37° C. for 24 hours to 80 hours to obtain the second culture medium containing polyketide compounds or derivatives thereof.

In accordance with some embodiments, the second culture medium may include M9 minimal medium, LB medium, TB medium or a combination thereof, but it is not limited thereto. In accordance with some embodiments, the second culture medium may include appropriate antibiotics, so that plasmids can be selected based on the antibiotics they tolerate. In accordance with some embodiments, the second culture medium may further include yeast extract, sodium glutamate, glucose, calcium chloride (CaCl₂)), magnesium sulfate (MgSO₄), rare trace metals, or other suitable nutrients. The second culture medium can be used for fermentation culture of the genetically modified microorganism. In accordance with some embodiments, the pH value of the second culture medium may be between pH 6 and pH 8, for example, pH 6.2, pH 6.5, pH 6.8, pH 7, pH 7.2, pH 7.5 or pH 7.8, but it is not limited thereto. In accordance with some embodiments, the temperature range for culturing the genetically modified microorganism in the second culture medium may be, for example, 28° C., 29° C., 30° C., 31° C., 32° C., 33° C., 34° C., 35° C., 36° C. or 37° C., but it is not limited thereto. Furthermore, in accordance with some embodiments, the time period for culturing the genetically modified microorganism in the second culture medium may be 24 hours, 28 hours, 30 hours, 35 hours, 40 hours, 45 hours, 50 hours, 55 hours, 60 hours, 65 hours, 70 hours, 75 hours or 80 hours, but it is not limited thereto.

Moreover, the method for producing a polyketide compound or a derivative thereof may include the step (d) of separating the polyketide compound or derivatives thereof from the second culture medium of step (c).

The polyketide compound or derivatives thereof can be separated and purified from the second culture medium by any suitable method. In accordance with some embodiments, the polyketide compound may include flavokermesic acid, but it is not limited thereto. In accordance with some embodiments, the derivative of the polyketide compound may include carminic acid, but it is not limited thereto.

In order to make the above and other purposes, features, and advantages of the present disclosure more obvious and easy to understand, several embodiments, comparative examples, and test examples are given below, and are described in detail as follows, but they are not intended to limit the scope of the present disclosure.

### Example 1—Establishing a Strain that has the Pta Gene Deleted and Includes Exogenous Nucleotide Sequences Encoding Transaminase and Reductase

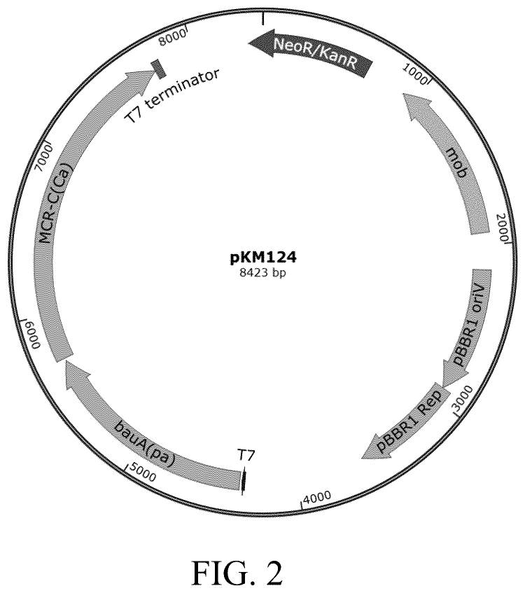

A plasmid pKM124 was constructed by gene modification, in which the nucleotide sequence encoding β-alanine-pyruvate transaminase (BauA) (SEQ ID NO: 4) and the nucleotide sequence encoding methyl coenzyme M reductase operon protein C (MCR-C) (SEQ ID NO: 6) were carried. The structure of the constructed plasmid is shown in FIG. 2.

The plasmid pKM124 was transformed into an Escherichia coli BL-21 strain carrying a polyketide metabolic pathway by electroporation. The strain carried three plasmids, pKM124, pFA and pCA. The pKM124 plasmid carried a nucleotide sequence encoding BauA (SEQ ID NO: 4) and a nucleotide sequence encoding MCR-C(SEQ ID NO: 6). The pFA plasmid carried nucleotide sequences encoding AntDEFGB (SEQ ID NOs: 9 to 13), a nucleotide sequence encoding ZhuI (SEQ ID NO: 7) and a nucleotide sequence encoding ZhuJ (SEQ ID NO: 8). The pCA plasmid carried a nucleotide sequence encoding DnrF^{P217K} (SEQ ID NO: 14) and a nucleotide sequence encoding GtCGT^{V93Q/Y193F} (SEQ ID NO: 15). A single colony of the above-mentioned transformed strain growing on a plate was inoculated into 2 mL of LB (Lysogeny broth) medium supplemented with appropriate antibiotics, and cultured at a temperature of 28° C. to 37° C. and a rotation speed of 150 rpm to 250 rpm for 12 to 24 hours to obtain a seed culture medium. 0.1% to 5% of the seed culture medium was inoculated into a fermentation medium (M9 medium supplemented with 2-20 g/L yeast extract, 0-10 g/L sodium glutamate, 5-20 g/L glucose, 100 μM CaCl₂, 1 mM MgSO₄, rare trace metals and appropriate antibiotics) in a 250 mL triangular shake flask, and fermentation production was carried out in an incubator at 28° C. to 37° C. and a rotation speed of 150 rpm to 250 rpm. When the strain concentration grew to an absorbance value (OD600) greater than 0.3, 0.1 mM to 1 mM IPTG was added to the culture medium to induce protein production. The fermentation was then continued for 72 hours and samples were taken for analysis.

The solids in the sample were first removed by centrifugation, and then the supernatant was filtered through a 0.22 μm filter membrane, and the product was analyzed by high performance liquid chromatography (HPLC) equipment (SHIMADZU, LC-20A series). The analysis conditions of HPLC were set as follows:
Mobile phase: acetonitrile and 1-5% acetic acid ratio=40/60
Flow rate: 0.3-1 mL/min
Temperature: 35-45° C.
Detector: UV light
Wavelength: 460 nm

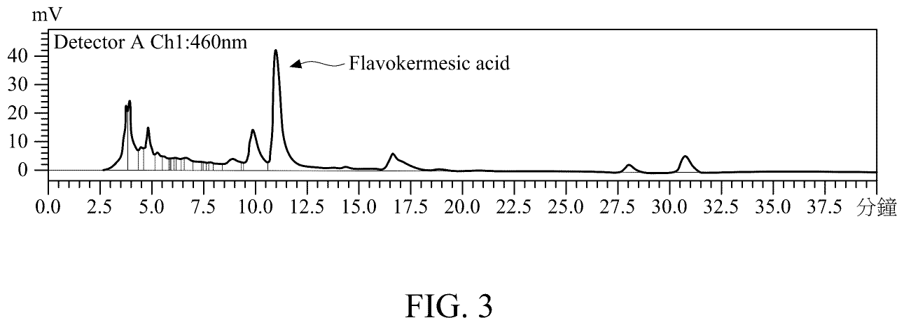

The HPLC analysis of the product produced by the aforementioned strain after fermentation is shown in FIG. 3. As shown in FIG. 3, the product produced by the aforementioned strain via the polyketide anabolism pathway include polyketide compounds, such as flavokermesic acid.

### Example 2—Testing the Effect of Pta Gene Deletion on the Production of Polyketides

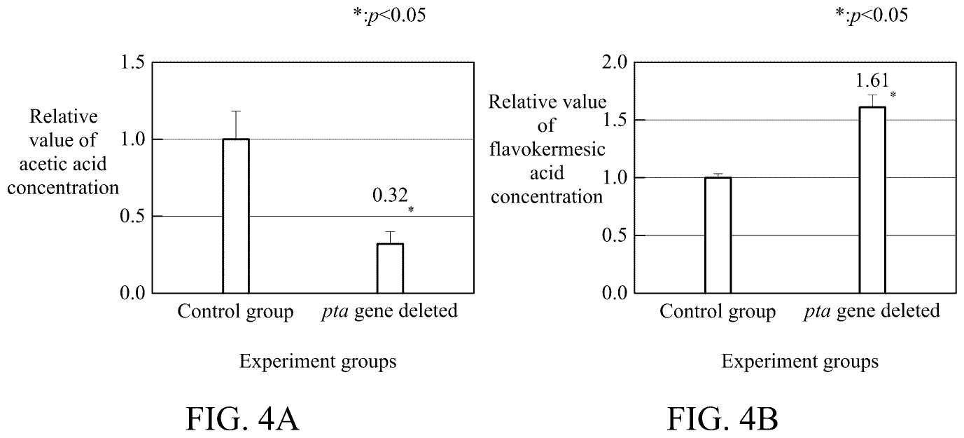

A strain with the nucleotide sequence of the pta gene (SEQ ID NO: 2) deleted alone was established by a method similar to that described in Example 1, and the relevant plasmids of the carmine metabolic pathway were transformed into the Escherichia coli BL-21 strain with the pta gene deleted. The strain in which the pta gene was deleted carried three plasmids, pACC, pFA and pCA. The pACC plasmid carried a nucleotide sequence encoding carboxylase AccBCD (SEQ ID NO: 16 and SEQ ID NO: 17, the nucleotide sequences shown in SEQ ID NO: 16 and SEQ ID NO: 17 encode the AccBC unit and AccD unit of carboxylase, respectively). The pFA plasmid carried nucleotide sequences encoding AntDEFGB (SEQ ID NOs: 9 to 13), a nucleotide sequence encoding zhuI (SEQ ID NO: 7) and a nucleotide sequence encoding zhuJ (SEQ ID NO: 8). The pCA plasmid carried a nucleotide sequence encoding DnrF^{P217K} (SEQ ID NO: 14) and a nucleotide sequence encoding GtCGT^{V93Q/Y193F} (SEQ ID NO: 15). Then, the strain was grown on a plate, a single colony was selected for fermentation culture, and the acetic acid and flavokermesic acid yields of the product were analyzed by HPLC. The results are shown in FIG. 4A and FIG. 4B.

FIG. 4A shows the effect of pta gene deletion on the acetic acid production of the strain, and FIG. 4B shows the effect of pta gene deletion on the polyketide derivative (flavokermesic acid) production of the strain. The “Control group” in the figure refers to a strain in which the pta gene is not deleted, and the “pta gene deleted” refers to a strain in which the pta gene is deleted.

As shown in FIG. 4A, in the strain group in which the pta gene was deleted, the production of acetic acid was reduced by about 68% compared to the wild type. As shown in FIG. 4B, in the strain group in which the pta gene was deleted, the production of flavokermesic acid increased by about 61% compared to the wild type. It can be seen from this that the deletion of the pta gene can reduce the metabolic pathway that consumes acetyl-CoA to produce acetate, increase the concentration of acetyl-CoA in the microorganism, and thus increase the production of flavokermesic acid.

### Example 3—Testing the Effect of Substituting Carboxylase with Transaminase and Reductase on the Production of Polyketide Compounds

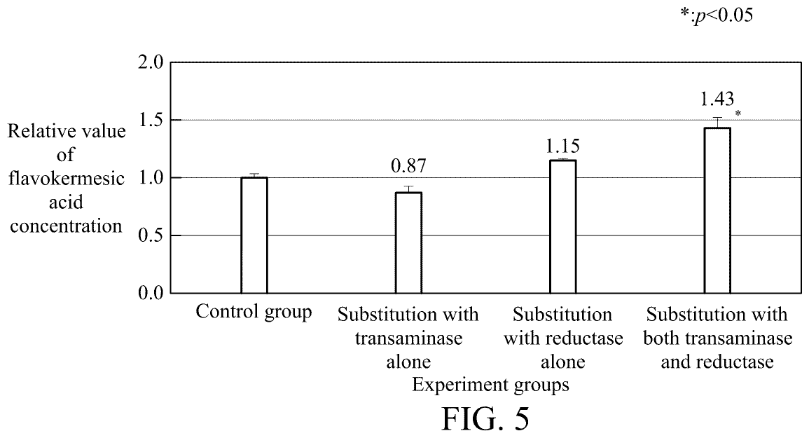

Using a method similar to that described in Example 1, strains carrying a nucleotide sequence encoding β-alanine-pyruvate transaminase (BauA) (SEQ ID NO: 4) alone, a nucleotide sequence encoding methyl-CoA reductase operon protein C (MCR-C) (SEQ ID NO: 6) alone, and nucleotide sequences of both BauA and MCR-C were respectively established, and the relevant plasmids of carmine metabolic pathway were transformed into Escherichia coli BL-21 strains. The strain carrying the nucleotide sequence encoding BauA alone carried three plasmids, pKM129, pFA and pCA. The pKM129 plasmid carried the nucleotide sequence encoding BauA (SEQ ID NO: 4). The pFA plasmid carried nucleotide sequences encoding AntDEFGB (SEQ ID NO: 9 to 13), the nucleotide sequence encoding ZhuI (SEQ ID NO: 7) and the nucleotide sequence encoding ZhuJ (SEQ ID NO: 8). The pCA plasmid carried the nucleotide sequence encoding DnrF^{P217K} (SEQ ID NO: 14) and the nucleotide sequence encoding GtCGT^{V93Q/Y193F} (SEQ ID NO: 15). The strain carrying the nucleotide sequence encoding MCR-C alone carried three plasmids pKM130, pFA and pCA. The pKM130 plasmid carried the nucleotide sequence encoding MCR-C(SEQ ID NO: 6). The pFA plasmid carried nucleotide sequences encoding AntDEFGB (SEQ ID NOs: 9 to 13), a nucleotide sequence encoding ZhuI (SEQ ID NO: 7) and a nucleotide sequence encoding ZhuJ (SEQ ID NO: 8). The pCA plasmid carried a nucleotide sequence encoding DnrF^{P217K} (SEQ ID NO: 14) and a nucleotide sequence encoding GtCGT^{V93Q/Y193F} (SEQ ID NO: 15). The strain carrying the nucleotide sequences of both BauA and MCR-C carried three plasmids, pKM124, pFA and pCA. The pKM124 plasmid carried the nucleotide sequence encoding BauA (SEQ ID NO: 4) and the nucleotide sequence encoding MCR-C (SEQ ID NO: 6). The pFA plasmid carried the nucleotide sequence encoding AntDEFGB (SEQ ID NOd: 9 to 13), the nucleotide sequence encoding ZhuI (SEQ ID NO: 7) and the nucleotide sequence encoding ZhuJ (SEQ ID NO: 8). The pCA plasmid carried the nucleotide sequence encoding DnrF^{P217K} (SEQ ID NO: 14) and the nucleotide sequence encoding GtCGT^{V93Q/Y193F} (SEQ ID NO: 15). Then, the strains were grown on a plate, a single colony was selected for fermentation culture, and the product was analyzed for the yield of flavokermesic acid by HPLC. The results are shown in FIG. 5.

FIG. 5 shows the results of testing the effect of substituting carboxylase with transaminase and/or reductase on the production of flavokermesic acid in the strain. The “Control group” in the figure refers to a strain in which the gene of carboxylase is not substituted with the gene of transaminase and/or reductase. The “Substitution with transaminase alone” refers to a strain in which the gene of carboxylase is substituted with the gene of transaminase. The “Substitution with reductase alone” refers to a strain in which the gene of carboxylase is substituted with the gene of reductase. The “Substitution with both transaminase and reductase” refers to a strain in which the gene of carboxylase is substituted with the genes of transaminase and reductase.

As shown in FIG. 5, the expression of transaminase or reductase alone instead of carboxylase has little effect on the yield of flavokermesic acid produced by the strain. It should be noted that, compared with the wild type, the production of flavokermesic acid in the strain co-expressing transaminase and reductase instead of carboxylase can be increased by about 43.9%. It can be seen from this that the combination of transaminase and reductase can replace carboxylase. In this way, acetyl-CoA can be converted into malonyl-CoA without being affected by the feedback inhibition of the carboxylase by its product malonyl-CoA. Therefore, the concentration of malonyl-CoA in the genetically modified microorganism can be increased, thereby increasing the production of flavokermesic acid.

### Example 4—Testing the Effect of Substituting Carboxylase with Transaminase and Reductase and Deleting the Pta Gene on the Production of Polyketide Compounds

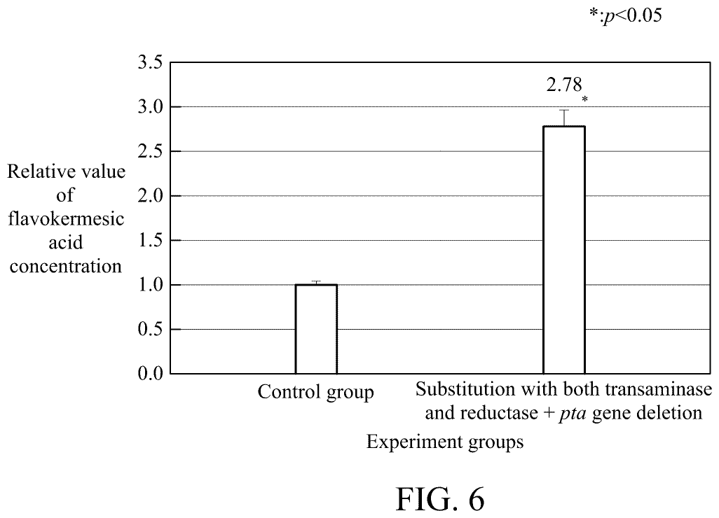

Using a method similar to that described in Example 1, a plasmid with a nucleotide sequence of the pta gene (SEQ ID NO: 2) deleted and carrying a nucleotide sequence encoding β-alanine-pyruvate transaminase (BauA) (SEQ ID NO: 4) and a nucleotide sequence encoding methyl coenzyme M reductase operon protein C (MCR-C) (SEQ ID NO: 6) was established. The plasmid was transferred to the Escherichia coli BL-21 strain in which the pta gene was deleted. The strain in which the pta gene was deleted carried three plasmids pKM124, pFA and pCA. The pKM124 plasmid carried a nucleotide sequence encoding BauA (SEQ ID NO: 4) and a nucleotide sequence encoding MCR-C(SEQ ID NO: 6). The pFA plasmid carried nucleotide sequences encoding AntDEFGB (SEQ ID NOs: 9 to 13), a nucleotide sequence encoding ZhuI (SEQ ID NO: 7) and a nucleotide sequence encoding ZhuJ (SEQ ID NO: 8). The pCA plasmid carried a nucleotide sequence encoding DnrF^{P217K} (SEQ ID NO: 14) and a nucleotide sequence encoding GtCGT^{V93Q/Y193F} (SEQ ID NO: 15). Then, the strains were grown on a plate, a single colony was selected for fermentation culture, and the product was analyzed for the yield of flavokermesic acid by HPLC. The results are shown in FIG. 6.

FIG. 6 shows the results of testing the effects of substituting carboxylase with transaminase and reductase and deleting the pta gene on the production of flavokermesic acid in the strain. The “Control group” in the figure refers to a strain in which the pta gene is not deleted and the carboxylase gene is not substituted with the transaminase and/or reductase gene, and the “Substitution with both transaminase and reductase+pta gene deletion” refers to a strain in which the transaminase and reductase genes are co-expressed to substitute the carboxylase and the pta gene is deleted.

As shown in FIG. 6, compared to the wild type, the production of flavokermesic acid in the strain in which transaminase and reductase are co-expressed to substitute carboxylase and the pta gene is deleted can be increased by about 2.78 times. It can be seen that substituting carboxylase with a combination of transaminase and reductase and deleting the pta gene can increase the concentrations of malonyl-CoA and acetyl-CoA in the genetically modified microorganisms, thereby increasing the production of flavokermesic acid.

To summarize the above, in accordance with some embodiments of the present disclosure, the provided genetically modified microorganism can block the metabolic pathway that consumes acetyl-CoA to generate acetate, thereby increasing the concentration of acetyl-CoA in the microorganism, and can produce malonyl-CoA in the metabolic pathway without feedback inhibition of enzyme activity. Therefore, the concentration of malonyl-CoA in the microorganism can be increased, thereby increasing the production of polyketide compounds (e.g., flavokermesic acid) or their related derivatives (e.g., carminic acid).

Although some embodiments of the present disclosure and their advantages have been described as above, it should be understood that various changes, substitutions and alterations can be made herein without departing from the spirit and scope of the disclosure as defined by the appended claims. In addition, each claim constitutes an individual embodiment, and the claimed scope of the present disclosure also includes the combinations of the claims and embodiments. The scope of protection of the present disclosure is subject to the definition of the scope of the appended claims.

1. A genetically modified microorganism for increasing the production of a polyketide compound or a derivative thereof, comprising any one or both of the following genetic modifications:
(a) a deleted endogenous pta gene encoding a phosphate acetyltransferase, wherein an expression level of the phosphate acetyltransferase of the deleted endogenous pta gene is lower than an expression level of its wild type; and
(b) an added first exogenous nucleotide sequence and second exogenous nucleotide sequence, wherein the first exogenous nucleotide sequence encodes a transaminase and the second exogenous nucleotide sequence encodes a reductase.

2. The genetically modified microorganism as claimed in claim 1, wherein the transaminase comprises β-alanine-pyruvate transaminase (BauA).

3. The genetically modified microorganism as claimed in claim 1, wherein the reductase comprises Methyl coenzyme M reductase operon protein C (MCR-C).

4. The genetically modified microorganism as claimed in claim 1, wherein the source of the genetically modified microorganism comprises bacteria.

5. The genetically modified microorganism as claimed in claim 4, wherein the bacteria comprises Escherichia coli, Corynebacterium glutamicum, Bacillus subtilis, Pseudomonas putida, Yarrowia lipolytica, Saccharomyces cerevisiae or Pichia pastoris.

6. The genetically modified microorganism as claimed in claim 1, further comprising the following genetic modifications:
(c) an added third exogenous nucleotide sequence, wherein the third exogenous nucleotide sequence encodes an enzyme related to carmine synthesis.

7. The genetically modified microorganism as claimed in claim 6, wherein the enzyme related to carmine synthesis comprises at least one of the following: cyclase ZhuI, aromatase ZhuJ, type II polyketide synthase complex AntDEFGB, hydroxylase DnrF^{P217K} glucosyltransferase GtCGT^{V93Q/Y193F}, glucosyltransferase UGT2, monooxygenase AptC and 4′-phosphopantetheinyl transferase NpgA.

8. The genetically modified microorganism as claimed in claim 1, wherein the polyketide compound comprises flavokermesic acid.

9. The genetically modified microorganism as claimed in claim 8, wherein compared to its wild type, the production of flavokermesic acid in the genetically modified microorganism is increased by 1.5 to 5 times.

10. The genetically modified microorganism as claimed in claim 1, wherein the derivative of the polyketide compound comprises carminic acid.

11. The genetically modified microorganism as claimed in claim 1, which has a deposit number BCRC 940701.

12. A method for producing a polyketide compound or a derivative thereof, comprising the following steps:
(a) providing a genetically modified microorganism as claimed in claim 1;
(b) providing a first culture medium, inoculating the genetically modified microorganism into the first culture medium, and culturing the genetically modified microorganism at a temperature of 28° C. to 37° C. for 12 hours to 24 hours;
(c) inoculating the first culture medium containing the cultured genetically modified microorganism into a second culture medium, and culturing the genetically modified microorganism at a temperature of 28° C. to 37° C. for 24 hours to 80 hours to obtain the second culture medium containing a polyketide compound or a derivative thereof; and
(d) separating the polyketide compound or the derivative thereof from the second culture medium of step (c).

13. The method for producing a polyketide compound or a derivative thereof as claimed in claim 12, wherein the pH value of the first culture medium is between pH 6 and pH 8, and the pH value of the second culture medium is between pH 6 and pH 8.

14. The method for producing a polyketide compound or a derivative thereof as claimed in claim 12, wherein the first culture medium comprises LB medium, NB medium, M9 medium, TB medium or a combination thereof.

15. The method for producing a polyketide compound or a derivative thereof as claimed in claim 12, wherein the second culture medium comprises M9 minimal medium, LB medium, TB medium or a combination thereof.

16. A novel genetically modified Escherichia coli strain whose deposit number is BCRC 940701, wherein an endogenous pta gene of the novel genetically modified Escherichia coli strain is deleted and it comprises a first exogenous nucleotide sequence and a second exogenous nucleotide sequence, the endogenous pta gene encodes a phosphate acetyltransferase, the first exogenous nucleotide sequence encodes a transaminase, and the second exogenous nucleotide sequence encodes a reductase.


SEQUENCE LISTING (17 sequences)

SEQ ID NO: 1 - protein, 714 aa, Escherichia coli
MSRIIMLIPTGTSVGLTSVSLGVIRAMERKGVRLSVFKPIAQPRTGGDAPDQTTTIVRANSSTTTAAEPLKMSYVEGLLSSNQKDVLMEEIVANYHANTKDAEVVLVEGLVPTRKHQFAQSLNYEIAKTLNAEIVFVMSQGTDTPEQLKERIELTRNSFGGAKNTNITGVIVNKLNAPVDEQGRTRPDLSEIFDDSSKAKVNNVDPAKLQESSPLPVLGAVPWSFDLIATRAIDMARHLNATIINEGDINTRRVKSVTFCARSIPHMLEHFRAGSLLVTSADRPDVLVAACLAAMNGVEIGALLLTGGYEMDARISKLCERAFATGLPVFMVNTNTWQTSLSLQSFNLEVPVDDHERIEKVQEYVANYINADWIESLTATSERSRRLSPPAFRYQLTELARKAGKRIVLPEGDEPRTVKAAAICAERGIATCVLLGNPAEINRVAASQGVELGAGIEIVDPEVVRESYVGRLVELRKNKGMTETVAREQLEDNVVLGTLMLEQDEVDGLVSGAVHTTANTIRPPLQLIKTAPGSSLVSSVFFMLLPEQVYVYGDCAINPDPTAEQLAEIAIQSADSAAAFGIEPRVAMLSYSTGTSGAGSDVEKVREATRLAQEKRPDLMIDGPLQYDAAVMADVAKSKAPNSPVAGRATVFIFPDLNTGNTTYKAVQRSADLISIGPMLQGMRKPVNDLSRGALVDDIVYTIALTAIQSAQQQ

SEQ ID NO: 2 - DNA, 2145 bp, Escherichia coli
  source 1..2145: mol_type = other DNA
atgtcccgtattattatgctgatccctaccggaaccagcgtcggtctgaccagcgtcagccttggcgtgatccgtgcaatggaacgcaaaggcgttcgtctgagcgttttcaaacctatcgctcagccgcgtaccggtggcgatgcgcccgatcagactacgactatcgtgcgtgcgaactcttccaccacgacggccgctgaaccgctgaaaatgagctacgttgaaggtctgctttccagcaatcagaaagatgtgctgatggaagagatcgtcgcaaactaccacgctaacaccaaagacgctgaagtcgttctggttgaaggtctggtcccgacacgtaagcaccagtttgcccagtctctgaactacgaaatcgctaaaacgctgaatgcggaaatcgtcttcgttatgtctcagggcactgacaccccggaacagctgaaagagcgtatcgaactgacccgcaacagcttcggcggtgccaaaaacaccaacatcaccggcgttatcgttaacaaactgaacgcaccggttgatgaacagggtcgtactcgcccggatctgtccgagattttcgacgactcttccaaagctaaagtaaacaatgttgatccggcgaagctgcaagaatccagcccgctgccggttctcggcgctgtgccgtggagctttgacctgatcgcgactcgtgcgatcgatatggctcgccacctgaatgcgaccatcatcaacgaaggcgacatcaatactcgccgcgttaaatccgtcactttctgcgcacgcagcattccgcacatgctggagcacttccgtgccggttctctgctggtgacttccgcagaccgtcctgacgtgctggtggccgcttgcctggcagccatgaacggcgtagaaatcggtgccctgctgctgactggcggttacgaaatggacgcgcgcatttctaaactgtgcgaacgtgctttcgctaccggcctgccggtatttatggtgaacaccaacacctggcagacctctctgagcctgcagagcttcaacctggaagttccggttgacgatcacgaacgtatcgagaaagttcaggaatacgttgctaactacatcaacgctgactggatcgaatctctgactgccacttctgagcgcagccgtcgtctgtctccgcctgcgttccgttatcagctgactgaacttgcgcgcaaagcgggcaaacgtatcgtactgccggaaggtgacgaaccgcgtaccgttaaagcagccgctatctgtgctgaacgtggtatcgcaacttgcgtactgctgggtaatccggcagagatcaaccgtgttgcagcgtctcagggtgtagaactgggtgcagggattgaaatcgttgatccagaagtggttcgcgaaagctatgttggtcgtctggtcgaactgcgtaagaacaaaggcatgaccgaaaccgttgcccgcgaacagctggaagacaacgtggtgctcggtacgctgatgctggaacaggatgaagttgatggtctggtttccggtgctgttcacactaccgcaaacaccatccgtccgccgctgcagctgatcaaaactgcaccgggcagctccctggtatcttccgtgttcttcatgctgctgccggaacaggtttacgtttacggtgactgtgcgatcaacccggatccgaccgctgaacagctggcagaaatcgcgattcagtccgctgattccgctgcggccttcggtatcgaaccgcgcgttgctatgctctcctactccaccggtacttctggtgcaggtagcgacgtagaaaaagttcgcgaagcaactcgtctggcgcaggaaaaacgtcctgacctgatgatcgacggtccgctgcagtacgacgctgcggtaatggctgacgttgcgaaatccaaagcgccgaactctccggttgcaggtcgcgctaccgtgttcatcttcccggatctgaacaccggtaacaccacctacaaagcggtacagcgttctgccgacctgatctccatcgggccgatgctgcagggtatgcgcaagccggttaacgacctgtcccgtggcgcactggttgacgatatcgtctacaccatcgcgctgactgcgattcagtctgcacagcagcagtaa

SEQ ID NO: 3 - protein, 448 aa, Pseudomonas aeruginosa
MNQPLNVAPPVSSELNLRAHWMPFSANRNFQKDPRIIVAAEGSWLTDDKGRKVYDSLSGLWTCGAGHSRKEIQEAVARQLGTLDYSPGFQYGHPLSFQLAEKIAGLLPGELNHVFFTGSGSECADTSIKMARAYWRLKGQPQKTKLIGRARGYHGVNVAGTSLGGIGGNRKMFGQLMDVDHLPHTLQPGMAFTRGMAQTGGVELANELLKLIELHDASNIAAVIVEPMSGSAGVLVPPVGYLQRLREICDQHNILLIFDEVITAFGRLGTYSGAEYFGVTPDLMNVAKQVTNGAVPMGAVIASSEIYDTFMNQALPEHAVEFSHGYTYSAHPVACAAGLAALDILARDNLVQQSAELAPHFEKGLHGLQGAKNVIDIRNCGLAGAIQIAPRDGDPTVRPFEAGMKLWQQGFYVRFGGDTLQFGPTFNARPEELDRLFDAVGEALNGIA

SEQ ID NO: 4 - DNA, 1353 bp, Pseudomonas aeruginosa
  source 1..1353: mol_type = other DNA
atgaaccagccgctgaacgttgcacctccggtatccagcgaactgaacttgcgtgctcactggatgccgttctctgcgaaccgtaacttccagaaagacccgcgtatcatcgttgcagctgaaggttcttggctgaccgacgacaaaggccgtaaagtttacgactctctgtctggtctgtggacctgtggtgcaggtcacagccgtaaagagatccaagaagctgttgctcgtcagttgggtactctggactactctccgggtttccagtacggtcatccgctgagcttccagctggcggagaagatcgctggcttgttgccgggtgaactgaaccacgtgttcttcaccggtagcggttctgaatgcgcggatacctccatcaagatggctcgtgcatactggcgtctgaaaggtcagccacagaaaaccaaactgatcggtcgtgcacgtggttaccacggcgttaacgttgcgggtacctctctgggtggtatcggtggtaaccgtaagatgttcggtcagctgatggacgttgatcacctgccgcacactctgcagccgggtatggcattcactcgtggtatggcgcagaccggtggtgtagaactggcgaacgaactgctgaagctgatcgaactgcacgacgcttccaacatcgctgcggttatcgttgaaccaatgtctggctctgcaggtgtgttggttccgccggtaggttacctgcagcgtctgcgtgaaatctgcgatcagcacaacatcttgctgatcttcgatgaagtgatcaccgcgttcggtcgtctgggtacctactctggcgcagaatacttcggtgttactccggacctgatgaacgttgcgaaacaggtgactaacggtgctgttccgatgggtgcagttatcgcatctagcgaaatctacgacaccttcatgaaccaggcactgccagaacatgctgttgagttcagccacggttacacctactccgcacatccggttgcttgtgcagctggtctggctgcactggacattctggcgcgtgacaacctggttcagcagtccgctgaactggcaccacacttcgagaaaggtctgcacggtttgcagggtgcaaagaacgttatcgacatccgtaactgcggcctggctggtgctatccagatcgcaccacgtgatggtgacccaaccgttcgtccattcgaagcaggtatgaaactgtggcagcagggcttctacgttcgtttcggtggtgatactctgcagttcggtccgactttcaacgcacgtccggaagaactggaccgtctgttcgatgctgttggtgaagcactgaacggtatcgctctcgagtaa

SEQ ID NO: 5 - protein, 674 aa, Chloroflexus aurantiacus
MADLSATTGARSASVGWAESLIGLHLGKVALITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLILENKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRVVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARWIALSDGARLALVTPETTATSTTEQFALANFIKTTLHAFTATIGVESERTAQRILINQVDLTRRARAEEPRDPHERQQELERFIEAVLLVTAPLPPEADTRYAGRIHRGRAITV

SEQ ID NO: 6 - DNA, 2031 bp, Chloroflexus aurantiacus
  source 1..2031: mol_type = other DNA
atggcagacctgtctgctaccaccggcgcacgttctgcgtctgttggttgggcggaatctctgatcggtctgcacctgggcaaagtagcgctgatcaccggtggttctgcgggtatcggtggtcagatcggtcgtctgctggcgctgtctggtgcacgtgtgatgctggcagctcgtgatcgtcacaaactggaacagatgcaggcgatgatccagtctgaactggcagaagttggttacaccgatgtggaagatcgtgttcacatcgctccaggttgcgatgtgtctagcgaagctcagctggctgacctggttgaacgtactctgtctgctttcggtaccgtagactacctgatcaacaacgcaggtatcgctggcgttgaagaaatggttatcgatatgccggttgaaggttggcgtcacactctgttcgcgaacctgatctccaactactctctgatgcgtaaactggcaccactgatgaagaaacagggttctggttacatcctgaacgtaagctcttacttcggtggtgagaaagacgcagcaatcccgtatccgaaccgtgctgactacgctgtttccaaagctggtcagcgtgcgatggcagaagtgttcgctcgctttctgggtccggaaatccagatcaacgcaatcgcaccaggtccggttgaaggcgaccgtctgcgtggtaccggtgaacgtccgggcctgttcgcgcgtcgtgcgcgtctgatcttggagaacaaacgtctgaacgaactgcacgctgctctgatcgcggctgctcgtaccgatgaacgttctatgcacgaactggttgaattgctgttgccgaacgatgttgctgctctggaacagaatccggcagcgccaaccgctttgcgtgaactggctcgtcgtttccgtagcgaaggtgatccggctgctagttccagctctgctctgctgaaccgttccatcgctgcgaaactgctggctcgtctgcacaacggtggttacgttctgccagctgatatcttcgcgaatctgccgaatccgccagatccgttcttcactcgtgctcagatcgaccgtgaagcacgtaaagttcgtgacggtatcatgggtatgctgtacctgcagcgtatgccgaccgaatttgacgttgcaatggcgaccgtttactacctggcagatcgtgttgtgagcggtgaaaccttccatccatctggtggtctgcgttacgaacgtactccgactggtggtgaactgttcggtttgccgtccccggaacgtctggcagaactggtaggttccactgtgtacctgatcggtgaacacttgaccgaacacctgaacctgttggcacgtgcttacctggaacgttacggtgcgcgtcaggtagtgatgatcgtggaaactgaaactggtgctgaaaccatgcgtcgtttgctgcacgaccatgttgaagcgggtcgtctgatgaccatcgttgcaggtgatcagatcgaagcggcgatcgaccaggcgatcactcgttatggtcgtccgggtccggtggtttgcactccgttccgtccgctgccgactgttccgctggttggtcgtaaagattctgactggtctaccgtactgtccgaagctgagttcgctgaactgtgcgaacaccagctgactcaccacttccgtgttgcgcgttggatcgctctgtccgatggtgcgcgtttggcgctggttactccggaaaccaccgcaacctccaccactgaacagttcgctctggcgaacttcatcaagaccactctgcacgcgttcaccgcgaccatcggtgttgaatccgaacgcaccgctcagcgtatcttgatcaaccaggttgacctgactcgtcgtgcacgtgctgaagaaccgcgtgatccgcacgaacgtcagcaagaactggaacgtttcatcgaagctgtgcttctggtaaccgctccacttccaccggaagctgatactcgttacgctggtcgtatccaccgtggtcgtgcaatcaccgtgctcgagtaa

SEQ ID NO: 7 - DNA, 510 bp, synthetic construct
  source 1..510: mol_type = other DNA
atgcgtcatgtagagcatacagtcaccgttgcggccccagcagacttggtttgggaggtacttgccgatgtcttaggctatgctgacatcttcccaccgacggaaaaagttgaaattcttgaggaggggcaaggataccaggtagtgcgccttcacgtcgatgttgcgggtgagattaatacatggaccagtcgtcgcgatttagaccctgcgcgccgcgtaattgcttaccgccaacttgagacggctccgatcgtgggccacatgagcggggaatggcgtgctttcacactggatgccgaacgtacccaattagtcctgactcacgatttcgtaacccgtgcagccggggatgacggtttagtcgccggaaaattgaccccagatgaggcgcgcgaaatgttagaagcggtggtagaacgtaactctgtcgccgacttaaacgcggtccttggagaagctgagcgtcgcgtccgcgcagccggtggagttggtaccgtaactgcgtaa

SEQ ID NO: 8 - DNA, 771 bp, synthetic construct
  source 1..771: mol_type = other DNA
atgtcagggcgcaaaacctttttagacttaagttttgctacccgcgacacaccgtcggaggcgactccggtggtggtagatttgctggaccacgtaactggagccaccgtattaggattatcacctgaggatttccccgatggtatggctatttccaatgagaccgttacgttgacgacccacactggcacgcacatggatgcgccactgcactatggtcccttaagtgggggagttccggcaaagtcgattgaccaagtgcccttggaatggtgctatggacctggagttcgtttggatgttcgccacgtgccggcaggagatggtattactgtcgatcatttgaacgccgcgttggatgcagcagagcacgatttggcccccggtgacattgtgatgctgtggaccggcgcggacgctctgtggggaacccgcgaatacttgagcacgtttccggggttaactgggaaggggacacaatttttggtcgaggcgggtgttaaagtcattggcattgatgcatggggactggatcgcccgatggcagctatgatcgaagaataccgtcgtacgggcgataaaggagcattatggccggctcacgtctatggacgcacacgcgaatacctgcaattagagaagcttaataatttgggcgctttaccaggagctacagggtatgacatttcatgctttccggttgcggttgcaggcactggagctgggtggactcgtgtggtcgccgttttcgagcaagaggaagaggattaa

SEQ ID NO: 9 - DNA, 1287 bp, synthetic construct
  source 1..1287: mol_type = other DNA
atgataataaataacagaaatgaatctcaaccacgtagagttgtggtgacagggctaggtgttgtcgcaccgacaggtgttggcgttaatgaattttggaacaatattcataacggcaaatcgggggtaagtgaatatgagtggggaagaaaaaaatttggttttaaaagcggagcaataggaaaagttcacggtaacgatagcgatagcaaagagtttgtgctgaaaagtgagcgtaaatatcttgagtttgcgctagaagcctctgagatggcaatgcaagatgcaaatttaaaaccttcagacattgatggccggcgttttggcgttgcgatagcaacagcgattgccgatgctgcgggaatggaagagtgtttgctcaggatcaccaaagggggcaaagagaatattcatcctgatttaattaaatcagaggattatgacagctttgatttcagctctgccgccacctctgttgcgaaaaaatatggcgcatcgatgtccgtcagtaacatatcaactgggtgtgcggcaggacttgatgcattaggcattgcgatggagcatatccgttatggcagagcggatgtgatgctggctggcgccagtgaagcgccgctttgtccactttctatcggctcttttgaagctttaggggcgctatcatcaagagaattggaaaatcagcaagcagcgacttgtcctttttcccttgagcgggatggatttgtgattgctgaagggtgtggaatattaattttagagtcttatgaacatgctaagcagcgtggagcacatatctatgctgaattagcagggtatgcgtccgtgaataacgcttatcatatgaccgacttgcctgcggatggaatggcaatggcgcggtgcattgatatggcgttgaaggatgcccagatatcgccatcagcggtcaattatattagtgctcatggcagttctacggctcaaaatgatattaacgaatcaaatgcgattaaatttgttttgggagaaaatgcatttgatattccaattaactcattaaagtcaatgacaggtcatgctttagctgccgctaatgcgatcgagtctgtagcgttatgtctggaaatagaaaagcaatatattcatccaacaattaattatcaaacgccggaccctgattgcgatttagattatattcctaatcaaggttgcgcatatccaattaagaccgcattaaaattatcgagtggtttttctggtattcacagtgttattgttatgagggcagtagacaatgcgtaa

SEQ ID NO: 10 - DNA, 1116 bp, synthetic construct
  source 1..1116: mol_type = other DNA
atgcgtaaaagagttgttgttaccggcgttggcgcagtacatcctgatggcaatgatgtcaccgctataaaaacaaaagtgattcagaaattattgggtcaggaatcgataaataataccaacaaaagttctgtaataaggacattgaatgatttcgatggggcaaaatatatcaataaccgcttaagacgtaaaattgatgaattttcagtttatggtatcgtcgccgttgaaatggcattaaaagcgagcagattggatgtagataagcttgatcctaatcgtgttggcatatatgttggaaactgttttggcggatggcagcatattgaggatgaagttaaagcgctccatgttgaaggcatatcggggatgggaccttatgttgctacggcatggttccctgctgcgcttcaagggcaattgtcactgctttatggttttagtgcgcaatctaagacattttccacctccgatgtagcagggatgcaagcaataggctatgcggctgaagcgatttctaatggtgttgccgaagtgatgttatgtggcgcgtcagaacatctttccagcccgttagttaaaagtttactggagaaagagtcaagccagaaacactctgaggtttttggcgaaagacagccaggggacttttccgaaggcgctgcatttctagtgctggaagagaggcaacatgctttagaacgcggcgcttcgatattgtgtgaattaacgggttttgttgattatttttcaccggataaaaatacaagaaataacaccttagaatatactgctgaactattcaaccataatgagaatgctgtatttattatggatggaatatatgatgatgaaaaagaaataacgagtaaggctttctccaataaagagataaaaacatcatttataaatctgaggccttacttgaataatcaattttcagtcagcggcgtaattgattcagtcctggcatcatcatttttatcagaaaataacggggatggagaacaacaatctaataaaataaatgaactttcaaatactaaccaaataataattcagcgctttagtaaccagggtcatgtatgtgcgttgagtttttcagcaatttaa

SEQ ID NO: 11 - DNA, 249 bp, synthetic construct
  source 1..249: mol_type = other DNA
atgaataataacccagaagtaaaaataaaaacgattttgtctctttttcttaacgttaatattgatgatttcaatatggatgcaaaccttgctgatgcctatgatatggattctacggaattggctgacttggcaaaagagattacgaaagagttcggtatttccgtgacgaaaagtcagttcagtcattgggaaacaggaagagccgttcttgatttcgtctcatcaagtttaaacgataaaaattaa

SEQ ID NO: 12 - DNA, 1548 bp, synthetic construct
  source 1..1548: mol_type = other DNA
atgaaactaatctctatgttgttacattcagagcatgataacttacatcatgattgtattgtcactaaggattatcattatacaagaaaagaggtgatatcttctgtttcccatttaattgatgatttattgagtcgaggagtgcaaaaaggtaataaagtcattgttatatttgaacatgatgaattaggtgttttctttttggctgccgccagtgctatggggttgcatttattaatgccctataatttatcatcagcgacaatcgatgaatggattaattttaccaatgaagtgcaatacgattttgttgtttatctcaaaaaagataaacattttgttggaaaattaaaagaaaacaacattaatgttattgatatttcagatcataagatcagagttagtgatgatattgcggaaatcccaatgataacttattctccgcaacctattgctaactttattgtcctgttcaccagtgggagtacaggcaaaccaaaagccattagtatttcagaatcgttagtatgtcgtcgaatttattcggtgaccgagaaattaaaatttacgcaagatgccaaaatattcatgtcaggtttgttgaataatacaactggagtgattttttctttcggctcattattgcatcaatcaacactttttatacccgaagatagaaatgtagagagatggcctgattatctttctcgcaataaaatcactcatattatgttacgcccagaatcaatgaaattattcgttaaatcgacagcagaacttaatattgatctctcttgtttacgggtggttgcttatggcgctgcggcgatgcctcctagcgtacttgagaaagggcgacaattaattggctgtgaatgggtgcagggatatgggttaagtgaaacttatggtcctttctgttgggtggatgagcaagatcatcgtgataaaagatatctcaattcaatttattgtgttggtaagattgataatacattggaagtggcagttaaacctattataggttcatcggataatatcggagaaattatactaaggggtaaaagtattatggaaggatattatgatgtcctttctggagaaataacgcctcctgatgaatggtttgccactggtgatcttggttatatagatgaagagggttatttagttttgaaaggacgtaagcaaaatacgtttatgagtgctaacggacacagaatttatcctgaagaaattgaatctattttatcccgaatacccaatgtgaatgtcgctacggttgttggtttttctttccatgaaaatggtgttgctattgatcagccggttgcttgcatgagtggagagatatctaagaagtcattacctgaaattgaagatattatttcatcatttttaatgagtaaactcagtcgagaaaaatggccggattggttctatgttactgatgaatgctttccgaaaagccataatgataagatattgaaatcagagttaattaaatcaatcgatcctaagaaattatttacattgaggaaccaataa

SEQ ID NO: 13 - DNA, 714 bp, synthetic construct
  source 1..714: mol_type = other DNA
atggacggtatttctttatcatctgatttttttgatctttggattatcaaaatcgacgatattgatttagcttctattgaacagttaattcactgttctgatatagttcgccataaccaaatttgtttagcggatagaagaaagagatttatatttagacgggctgcattacgttatgttttgagtcaatatttatctgattatgaaatcataacgaatgataacggaaaaccttatatatccacggagcaagacttcaaatattatttttcactgagtgcttcaggaaactattgtgccattggttttagctcaagggaaataggtgttgatattgaagtcactccttctaaggtaaaattttcagaaattattgaacgttttattaaggataaagatttggaatatatgaaaggtataatgttaaaacaactatcaggagttagtctcggatttaataactattatcatttaatgtcattatattattgggttagacttgaagcatatattaaattatttgcttcgactttacatgagaaattattggttaataactctgattctgttaaagatatgaaagaattggaggcaagcacattattgattcatagtcagcaatttgtttgtgccttatctcaaaagaaagtcatttctacaccaaatatcaaggaaataaattattccgaaattataaggaacaaagatgagtaa

SEQ ID NO: 14 - DNA, 1470 bp, synthetic construct
  source 1..1470: mol_type = other DNA
atggcactgacgaaaccggatgttgatgttctggttgttggtggtggtctgggtggtctgagcacggcgctgtttctggcgcgccgcggtgcacgtgttctgctggttgaacgtcatgcgagtactagcgttctgccgaaagcggcgggtcagaatccgcgtacaatggaactgtttcgttttggtggtgttgcagatgaaattctggcaaccgatgatattcgtggtgcacagggtgattttacaattaaagttgttgaacgtgtgggtggtcgtgttccggcgcagctgcgtgaatcttttgaagaactggtgggtgcaacagaacagtgtacaccgatgccgtgggccctggcaccgcaggatcgcgttgaaccggttctggttgcacatgcagcaaaacatggtgctgaaattcgttttgcaactgaactgacctcatttcaggcgggcgatgatggtgttaccgcacgtctgcgtgatctgggtaccggtgccgaaagtacagtcagcgcacgctatctggttgcagcagatggtccgcgtagcgccattcgtgaaagcctgggtattacccgtcatggccacggtaccctggcacattttatgggtgttatttttgaagcagatctgaccgccgttgttccgaaaggtagtacaggttggtattatctgcagcatcctgattttaccggtacctttggtccgaccgatcgtccgaatcgtcataccttttatgttcgttatgatccggaacgtggtgaacgtccggaagattatacgccgcagcgttgtaccgaactgattcgcctggcagttgatgcaccgggtctggttccggatattctggatattcaggcatgggatatggcggcttatattgcagatcgttggcgtgaaggtccggttctgctggtgggtgatgcagcaaaagttaccccgcctacaggtggtatgggtggtaataccgcaattggtcatggttttgatgttgcgtggaaactggccgcggttctgcgtggtgaagccggtgaacgtctgctggatagctatggtgccgatggcagtctggttagtcgtctggttgttgatgaaagcctggcgatttatgcacagcgtatggcaccgcatctgctgggtagtgttccggaagaacgtggtaccgcacaggttgttctgggttttcgttatcgttctactgcagttgcagcagaagatgatgatccggaaccgactgaagatcctcgtcgtcctagtggtcgtcctggttttcgtgcaccgcatgtttggattgaacaggatggtacccgtcgtagcacagtggaactgtttggtgattgttgggtgctgctggcagcgccggaaggtggtgcatggggtcaggcagctgctcgtgctgcacgtatttgggcaagtgcaagtacatctattagcagcgcggcaatgagtccgccgccgcctgctaattaa

SEQ ID NO: 15 - DNA, 1434 bp, synthetic construct
  source 1..1434: mol_type = other DNA
atgggcagcctgaccaacaacgataacctgcatatttttctggtttgttttatcggtcagggtgttgttaacccgatgctgcgtctgggtaaagcatttgcaagcaaaggtctgctggttacgctgtctgcaccggaaattgtgggtactgaaattcgtaaagccaataacctgaatgatgatcagccgattaaagttggtagcggtatgattcgttttgaattttttgatgatggctgggaatctgttaatggtagcaaaccgtttgatgtttggcagtatatcaaccatctggatcagacaggtcgtcagaaactgccaattatgctgaaaaagcatgaagaaacgggtacgccggtgagctgtctgattctgaatcctctggtgccgtgggttgccgatgtggcggatagtctgcagattccttgtgccaccctgtgggtacagagctgtgcaagctttagcgcatattatcattatcatcatggtctggttccgtttccgaccgaaagtgaaccggaaattgatgttcagctgccgggtatgccgctgctgaaatatgatgaagttccggattttctgcatcctcgtaccccgtatccgttttttggtaccaatattctgggtcagtttaaaaatctgtcaaaaaatttttgtattctgatggataccttttatgaactggaacatgaaattattgataatatgtgtaaactgtgtccgattaaaccgattggtccgctgtttaaaattccgaaagatccgagcagcaatggtattaccggtaattttatgaaagttgatgattgtaaagaatggctggatagccgtccgaccagcaccgttgtttatgttagcgttggtagcgttgtttatctgaaacaggaacaggttaccgaaatggcatacggtattctgaatagcgaagttagctttctgtgggttctgcgtccgccgagcaaacgtattggtaccgaaccgcatgttctgccggaagaattttgggaaaaagcaggtgatcgtggtaaagttgttcagtggagcccgcaggaacaggttctggcacatccggcaaccgttggttttctgacccattgtggttggaatagcacccaggaagcaattagcagcggtgttccggttattacctttccgcagtttggtgatcaggttaccaatgcaaaatttctggttgaagaatttaaagttggtgttcgtctgggtcgtggtgaactggaaaatcgtattattacccgtgatgaagttgaacgtgcactgcgtgaaattaccagcggtccgaaagcagaagaagttaaagaaaatgcactgaaatggaaaaaaaaagcagaagaaaccgttgcaaaaggtggttatagcgaacgtaatctggttggttttattgaagaagttgcacgtaaaaccggtaccaaataa

SEQ ID NO: 16 - DNA, 1776 bp, synthetic construct
  source 1..1776: mol_type = other DNA
atgtcagtcgagactaggaagatcaccaaggttcttgtcgctaaccgtggtgagattgcaatccgcgtgttccgtgcagctcgagatgaaggcatcggatctgtcgccgtctacgcagagccagatgcagatgcaccattcgtgtcatatgcagacgaggcttttgccctcggtggccaaacatccgctgagtcctaccttgtcattgacaagatcatcgacgcggcccgcaagtccggcgccgacgccatccaccctggctacggcttcctcgcagaaaacgctgacttcgcagaagcagtcatcaacgaaggcctgatctggattggaccttcacctgagtccatccgctccctcggcgacaaggtcaccgctcgccacatcgcagataccgccaaggctccaatggctcctggcaccaaggaaccagtaaaagacgcagcagaagttgtggctttcgctgaagaatttggcctcccaatcgccatcaaggcagctttcggtggcggcggacgtggcatgaaggttgcctacaagatggaagaagtcgctgacctcttcgagtccgcaacccgtgaagcaaccgcagcattcggccgcggcgagtgcttcgtggagcgctacctggacaaggcacgccacgttgaggctcaggtcatcgccgataagcacggcaacgttgttgttgccggaacccgtgactgctccctgcagcgccgtttccagaagctcgtcgaagaagcaccagcaccattcctcaccgatgaccagcgcgagcgtctccactcctccgcgaaggctatctgtaaggaagctggctactacggtgcaggcaccgttgagtacctcgttggctccgacggactgatctccttcctcgaggtcaacacccgcctccaggtggaacacccagtcaccgaagagaccaccggcatcgacctggtccgcgaaatgttccgcatcgcagaaggccacgagctctccatcaaggaagatccagctccacgcggccacgcatttgagttccgcatcaacggcgaagacgctggctccaacttcatgcctgcaccaggcaagatcaccagctaccgcgagccacagggcccaggcgtccgcatggactccggtgtcgttgaaggttccgaaatctccggacagttcgactccatgctggcaaagctgatcgtttggggcgacacccgcgagcaggctctccagcgctcccgccgtgcacttgcagagtacgttgtcgagggcatgccaaccgttatcccattccaccagcacatcgtggaaaacccagcattcgtgggcaacgacgaaggcttcgagatctacaccaagtggatcgaagaggtttgggataacccaatcgcaccttacgttgacgcttccgagctcgacgaagatgaggacaagaccccagcacagaaggttgttgtggagatcaacggccgtcgcgttgaggttgcactcccaggcgatctggcactcggtggcaccgctggtcctaagaagaaggccaagaagcgtcgcgcaggtggtgcaaaggctggcgtatccggcgatgcagtggcagctccaatgcagggcactgtcatcaaggtcaacgtcgaagaaggcgctgaagtcaacgaaggcgacaccgttgttgtcctcgaggctatgaagatggaaaaccctgtgaaggctcataagtccggtaccgtaaccggccttgctgtcgctgcaggcgagggtgtcaacaagggcgttgttctcctcgagatcaagtaa

SEQ ID NO: 17 - DNA, 1632 bp, synthetic construct
  source 1..1632: mol_type = other DNA
atgaccatttcctcacctttgattgacgtcgccaaccttccagacatcaacaccactgccggcaagatcgccgaccttaaggctcgccgcgcggaagcccatttccccatgggtgaaaaggcagtagagaaggtccacgctgctggacgcctcactgcccgtgagcgcttggattacttactcgatgagggctccttcatcgagaccgatcagctggctcgccaccgcaccaccgctttcggcctgggctctaagcgtccagcgaccgacggcatcgtgaccggctggggcaccattgatggacgcgaagtctgcatcttctcgcaggacggcaccgtattcggtggcgcgcttggtgaggtgtacggcgaaaagatgatcaagatcatggagctggcaatcgacaccggccgcccattgatcggtctttacgaaggcgctggcgctcgcattcaggacggcgctgtctccctggacttcatttcccagaccttctaccaaaacattcaggcttctggcgttatcccacagatctccgtcatcatgggcgcatgtgcaggtggcaacgcttacggcccagccctgaccgacttcgtggtcatggtggacaagacctccaagatgttcgttaccggcccagacgtgatcaagactgtcaccggcgaggaaatcactcaggaagagctcggcggagcaaccacccacatggtgaccgctggtaactcccactacaccgctgcaaccgatgaggaagcactggattgggtacaggacctggtgtccttcctcccatccaacaaccgctcttacgcaccgatggaagacttcgacgaggaagaaggcggcgttgaagaaaacatcaccgctgacgatctgaagctcgacgagatcatcccagattccgcgaccgttccttacgacgtccgcgatgtcatcgaatgcctcaccgacgatggcgaatacctggaaatccaggcagaccgcgcagaaaacgttgttattgcattcggccgcatcgaaggccagtccgttggatttgttgccaaccagccaacccagttcgctggctgcctggacatcgactcctctgagaaggcagctcgcttcgtccgcacctgcgacgcgttcaacatcccaatcgtcatgcttgtcgacgtccccggcttccttccaggcgcaggccaggagtatggtggcatcctgcgtcgtggcgcaaagctgctctacgcatacggcgaagcaaccgttccaaagatcaccgtcaccatgcgtaaggcttacggcggagcgtactgcgtgatgggatccaagggcttgggctctgacatcaaccttgcatggccaaccgcacagatcgccgtcatgggcgctgctggcgcagtcggattcatctaccgcaaggagctcatggcagctgacgccaagggcctcgacaccgtcgctctggctaagtccttcgagcgcgagtacgaagaccacatgctcaacccgtaccacgctgcagaacgtggcctgatcgacgccgtgatcctgccaagcgaaacccgcggacagatttcccgcaaccttcgcctgctcaagcacaagaacgtcactcgccctgctcgcaagcacggcaacatgccactgtaa